(19) United States
(12) Patent Application Publication
(10) Pub. No.: US 2006/0134736 A1
(43) Pub. Date: Jun. 22, 2006

(54) Human growth hormone conjugated with biocompatible polymer

(76) Inventors: John W. Jacobs, Irvine, CA (US); Shiliang Qin, Irvine, CA (US); Mingyu Zhang, Foothill Ranch, CA (US); Huaina Li, Irvine, CA (US); Myung-Ok Park, Seoul (KR)

Correspondence Address: KNOBBE MARTENS OLSON & BEAR LLP, 2040 MAIN STREET, FOURTEENTH FLOOR, IRVINE, CA, 92614, US

(21) Appl. No.: 11/314,926

(22) Filed: Dec. 20, 2005

Related U.S. Application Data
(63) Continuation-in-part of application No. 11/187,522, filed on Jul. 22, 2005.
Application No. 11/187,522 is a continuation-in-part of application No. 10/947,513, filed on Sep. 22, 2004.
Application No. 10/947,513 is a continuation-in-part of application No. PCT/KR04/00701, filed on Mar. 27, 2004.

(30) Foreign Application Priority Data
Mar. 28, 2003 (KR) 10-2003-0019734
Feb. 6, 2004 (KR) 10-2004-0007983

(51) Int. Cl. C12P 21/06; A61K 38/27; C07K 14/61

(52) U.S. Cl. 435/68.1; 530/399; 514/12

(57) ABSTRACT

The present invention relates to conjugates of biocompatible polymers and hGH, particularly PEG-hGH, where the activated biocompatible polymer is conjugated to a carboxyl group of hGH at a molar ratio of 2:1 or less, preferably 1:1, methods of preparation, and related pharmaceutical compositions. The PEG-hGH conjugates have up to 20% of the activity of the native hGH while the in vivo half life is increased 10 fold. The PEG-hGH conjugates may be used therapeutically to treat growth retardation or growth failure, especially short stature in children, and conditions related to aging.

### CROSS-REFERENCE TO RELATED APPLICATIONS

This application is a continuation-in-part of U.S. application Ser. No. 11/187,522, filed Jul. 22, 2005, which is a continuation-in-part of U.S. application Ser. No. 10/947,513, filed Sep. 22, 2004, which is a continuation-in-part of International Application No. PCT/KR2004/000701, filed Mar. 27, 2004 which designates the United States and claims priority to Korean Patent Application No. 10-2004-0007983, filed Feb. 6, 2004 and Korean Patent Application No. 10-2003-0019734, filed Mar. 28, 2003.

### REFERENCE TO SEQUENCE LISTING, TABLE, OR COMPUTER PROGRAM LISTING

A sequence listing is included as page 24.

### BACKGROUND OF THE INVENTION

1. Field of the Invention

Embodiments of the present invention relate to Human Growth Hormone (hGH) which is conjugated with a biocompatible polymer at a molar ratio of 1:1, methods of preparation thereof and pharmaceutical compositions and kits comprising the same. Therapeutic treatment methods are also disclosed.

2. Description of the Related Art

Human growth hormone (hGH) is a single polypeptide chain composed of 191 amino acids (Goeddel D V, et al. 1979. Nature 231:542-548; Pearlman R, et al. 1993. Stability and Characterization of Human Growth Hormone in, Stability and Characterization of Protein and Peptide Drugs: Case Histories, edited by Y J Wang and R Pearlman. Plenum Press, N.Y.). Endogenous growth hormone is responsible for stimulating normal skeletal, connective tissue, muscle, and organ growth in children and adolescents. It also plays an important role in adult metabolism. Somatropin (recombinant human Growth Hormone) binds to growth hormone (hGH) receptors and produces a variety of physiologic effects that promote growth. Many of the biological actions of growth hormone are mediated by insulin-like growth factor-1 acting directly on the responsive tissue (Clark R. 1997. Endocrine Reviews 18:157-179).

The primary structure of human growth hormone is shown in FIG. 1 below. Endogenous hGH is produced in the anterior pituitary gland. Human growth hormone was first isolated in 1956 and its structure was identified in 1972 (Pearlman R, et al. 1993. Stability and Characterization of Human Growth Hormone in, Stability and Characterization of Protein and Peptide Drugs: Case Histories, edited by Y J Wang and R Pearlman. Plenum Press, N.Y.). Prior to 1985, growth hormone was derived from human cadavers, but the cloning and expression of human growth hormone in the late 1970's led to the availability of several marketed hGH products that mimic all of the normal functions of endogenous hGH (Drake W M, et al. 2001. Endocrine Reviews 22:425-450). Genentech's PROTROPIN® was originally approved by the FDA in the mid 1980s for treating growth failure due to growth hormone deficiency. Since then other indications for the use of hGH have been approved including growth deficiency seen in chronic renal disease, or Turner's syndrome and for treating cachexia and AIDS wasting (Drake W M, et al. ibid.).

An important advance in growth hormone therapy would be the availability of a long-acting hGH product. Currently hGH must be injected six times a week in children and a once a week injection would have a huge market impact. Genentech has marketed a long-acting formulation for hGH (NUTROPIN-Depot) but withdrew it from the market due to poor sales. This was due to a widespread belief among pediatric endocrinologists that the depot form of hGH was not as effective in accelerating growth rate in children. Embodiments of the present invention are directed to a more attractive approach for a long-acting growth hormone by pegylation of the protein.

Conjugates of proteins or pharmaceutically active molecules such as hGH to biocompatible polymers afford great advantages when they are applied in vivo and in vitro. When covalently bonded to biocompatible polymers, biologically active materials can exhibit modified surface properties and solubility, and thus have increased solubility in water or organic solvents. Further, the presence of biocompatible polymers can make the proteins and/or polypeptides conjugated to them more stable in vivo, increase biocompatibility of the proteins and reduce immune response, and reduce the clearance rate of the proteins by the intestine, the kidney, the spleen, or the liver.

Previous attempts to pegylate hGH by Genentech resulted in hGH preparations that were only 1/400 as active as the native hGH protein. This precluded the clinical development of those earlier peg-growth hormones. If the biological activity of biologically active molecules such as hGH can be maintained after conjugation with the polymer at a desired ratio, and a homogenous species of site-specific conjugates can be obtained, clinical usefulness of molecules such as hGH will increase remarkably. The present invention addresses this problem. By keeping the ratio of biocompatible polymer/hGH to less than 2:1, preferably 1:1, an active hGH with improved stability has been obtained by the methods as described herein. Pegylated hGH is provided that has up to 20% of the specific activity of the unpegylated protein and an increased half life in vivo.

### SUMMARY OF THE INVENTION

Embodiments of the invention are directed towards conjugates of biocompatible polymer-human Growth Hormone (hGH), wherein the biocompatible polymer is conjugated to a carboxyl group of hGH at a molar ratio of 2:1 or less. Preferably, the biocompatible polymer is PEG-20000 or PEG-30000.

Preferably, the carboxyl group of hGH is the C-terminus of hGH. Preferably, the activity of the conjugate is 10-20% of the activity of an unconjugated hGH protein. More preferably, the biocompatible polymer is conjugated to the carboxyl group of the hGH at a molar ratio of 1:1. Yet more preferably, the biocompatible polymer is PEG.

Embodiments of the invention are directed to a pharmaceutical composition which includes a pharmaceutically acceptable amount of the biocompatible polymer-hGH conjugate and a pharmaceutically acceptable carrier.

Embodiments of the invention are directed to a method of preparing a conjugate of biocompatible polymer-hGH which includes one or more of the following steps:
(a) providing a purified hGH protein;
(b) activating a biocompatible polymer with the stepwise addition of a coupling reagent; and
(c) conjugating the activated biocompatible polymer to a carboxyl group of the hGH at a molar ratio of 2:1 or less. Preferably, the molar ratio of the hGH to the activated biocompatible polymer is 1:1 to 1:20, the ratio of the hGH to the coupling reagent is 1:1 to 1:50, and pH is in the range of 2 to 5. Preferably, the biocompatible polymer is activated with a reactive functional group which is able to react with a carboxylic acid and/or a reactive carbonyl group. Preferably, the biocompatible polymer is PEG-20000 or PEG-30000. Preferably, the carboxyl group is the C-terminus of hGH.

In preferred embodiments, the purified hGH is provided by a method which includes one or more of the following steps:
(i) producing hGH in a recombinant host;
(ii) concentrating hGH using ammonium sulfate; and
(iii) purifying the concentrated hGH by anionic ion exchange chromatography.
   In preferred embodiments, the chromatography is performed as a single step.

Preferred embodiments are directed to a method of treating growth failure or growth retardation by administering an effective amount of the biocompatible polymer-hGH conjugate to a patient in need thereof. Preferably, the biocompatible polymer is PEG. More preferably, the PEG-hGH is conjugated at a molar ratio of 1:1. Preferably, the conjugate has 10-20% of the activity of unconjugated hGH protein. In preferred embodiments, the growth failure or growth retardation is due to hormone deficiency, chronic renal disease, Turner's syndrome, cachexia or AIDS wasting. Preferably, the conjugate is administered in combination with a pharmaceutically acceptable carrier. In some preferred embodiments, the administration is done by injection. In alternate preferred embodiments, the administration is oral. Preferably, the composition is administered no more than twice per week to the patient in need thereof.

A preferred embodiment is directed to a method of treating short stature in children by administering an effective amount of a composition which includes PEG-hGH to a patient in need thereof at a frequency of no more than twice/week. More preferably, the PEG is conjugated to a C-terminus carboxyl group of hGH at a molar ratio of 1:1 and the PEG-hGH has 10-20% of the activity of unconjugated hGH.

Preferred embodiments are directed to methods of treating adverse effects associated with aging such as decrease in lean muscle, increase in blood pressure, increase in cholesterol, increase in body fat, loss of skin tone, and decrease in bone density by administering an effective amount of a composition which includes PEG-hGH to a patient in need thereof. Preferably, the PEG is conjugated to a carboxyl group of hGH at a molar ratio of 1:1.

Embodiments of the invention are directed to a kit which includes the biocompatible polymer-hGH conjugate, preferably in lyophilized form, a pharmaceutically acceptable carrier for reconstitution of the conjugate; and a delivery device for delivery of the reconstituted conjugate to a patient in need thereof.

Preferably, the kit also includes a skin antiseptic and an instruction sheet. Preferably, the instruction sheet directs administration of the biocompatible polymer-hGH conjugate composition no more than twice per week to the patient in need thereof, preferably once per week. In some preferred embodiments, the kit includes the biocompatible polymer-hGH conjugate preloaded in a syringe.

Further aspects, features and advantages of this invention will become apparent from the detailed description of the preferred embodiments which follow.

### BRIEF DESCRIPTION OF THE DRAWINGS

These and other feature of this invention will now be described with reference to the drawings of preferred embodiments which are intended to illustrate and not to limit the invention.

FIG. 1 shows the primary structure of human Growth Hormone (hGH).

FIG. 2 shows SDS-PAGE gel of SYNTROPIN drug substance through successive purification steps. Lane 1: MW marker; Lane 2: hGH conditioned medium; Lane 3: (NH₄)₂SO₄ precipitated hGH medium; Lane 4: hGH purified by Q-Sepharose column; Lane 5: hGH purified by Q-Sepharose and Phenyl-Sepharose columns.

FIG. 3 shows a densitometer scan of SDS PAGE gel of purified SYNTROPIN drug substance. 99.9% of the material is in peak # 2.

FIG. 4 shows the SDS-PAGE analysis of human growth hormone (hGH) after 1 step column chromatography. FIG. 4A shows the SDS-PAGE gel. FIG. 4B shows a densitometric scan of lane 4.

FIG. 5 shows an HPLC profile of PEGylation of hGH on a size exclusion column.

FIG. 6 shows HPLC profiles of purified mono- and di-PET-hGH on a size-exclusion column. FIG. 6A shows profile for mono-PEG-hGH. FIG. 6B shows profile for di-PEG-hGH.

FIG. 7 shows the biological activity of PEG-hGH by cell proliferation assay.

FIG. 8 shows PK study of PEG-hGH in rats (dose=200 μg/kg, s.c. injection).

FIG. 9 shows bioassay of hGH in cells expressing full-length hGH receptors.

FIG. 10 shows an animal study of hGH and PEG-hGH injected into hypophysectomized rats with weight gain of the animals monitored over a 28 day period.

FIG. 11 shows the effect of hGH and PEG-hGH at different dosages on body weights in hypophysectomized rats The data are expressed as mean +/− S.E.M. The positive control (G5) and test samples were administered as a single dose by s.c. injection. Vehicle control (G1) and positive control were administered by s.c. injection daily for 6 days. GI: Vehicle control (n=6); G2: positive control hGH, 5 μg/head, daily injection (n=9); G3: positive control hGH, 10 μg/head, daily injection (n=9); G4: positive control hGH, 30 μg/head, daily injection (n=9); G5: positive control hGH, 180 μg/head, single injection (n=9); G6: test sample PEG-hGH, 30 μg/head, single injection (n=9); G7: test sample PEG-hGH, 60 μg/head, single injection (n=9); G8 test sample PEG-hGH, 180 μg/head, single injection (n=9). FIG. 11A shows G1-G8 on a single graph. FIG. 11B shows only G1-G4. FIG. 11C shows only G1 and G5-G8.

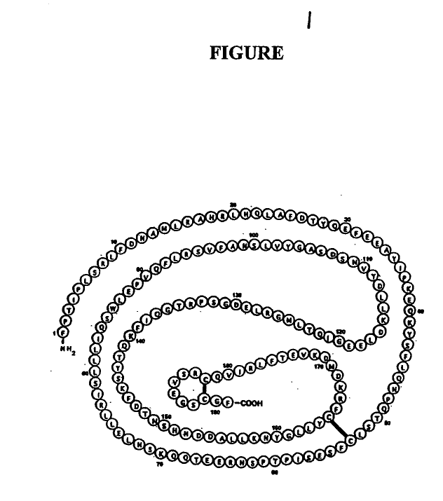

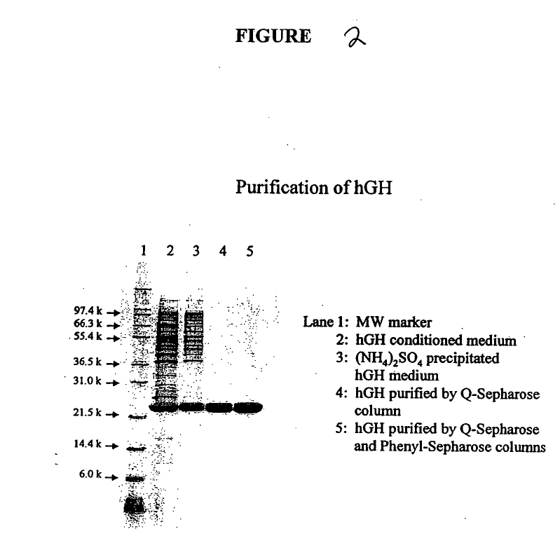

### DETAILED DESCRIPTION OF THE PREFERRED EMBODIMENT

While the described embodiment represents the preferred embodiment of the present invention, it is to be understood that modifications will occur to those skilled in the art without departing from the spirit of the invention. The scope of the invention is therefore to be determined solely by the appended claims.

Embodiments of the invention are directed to the conjugates of hGH with a biocompatible polymer, particularly PEG, where the activated biocompatible polymer is conjugated to a carboxyl group of biologically active hGH at a molar ratio of 2:1 or less, preferably 1:1. In preferred embodiments, pegylation is carried out as described in U.S. patent application Ser. No. 10/947,513, which is incorporated herein by reference. Briefly, a coupling agent such as EDAC is added stepwise to hGH and the biocompatible polymer at a pH between 2 and 5, preferably ≦3.0.

In another aspect, embodiments of the present invention relate to a pharmaceutical composition comprising a pharmaceutically acceptable amount of the conjugate, wherein the biocompatible polymer is conjugated to the C-terminus of the biologically active hGH at a molar ratio of 2:1 or less, preferably 1:1 and pharmaceutically acceptable carriers.

Embodiments of the invention are directed to a method of preparation of a conjugate of biocompatible polymer-biologically active hGH at the C-terminus of the biologically active hGH with a molar ratio of 2:1 or less, preferably 1:1. Biologically active hGH is produced by a recombinant method and purified using ammonium sulfate precipitation and chromatography. The purified hGH is conjugated to the activated biocompatible polymer with the stepwise addition of coupling reagent under conditions where the molar ratio of biologically active hGH to the activated biocompatible polymer is 1:1 to 1:20, the ratio of biologically active hGH to the coupling reagent is 1:1 to 1:50, and pH is in the range of 2 to 5.

Biocompatible Polymers

The term “conjugating material” used for conjugation of biologically active molecules means any biocompatible polymer which can be linked to biologically active molecules such as natural or synthetic polymers.

The term “biocompatibility” means biocompatible with living tissues or systems, and being nontoxic, noninflammatory, and noncarcinogenic without causing harm, inflammation, immune response and/or carcinogenesis in the body.

Biocompatible polymers are conjugated with biologically active materials such as hGH. The useful polymers of the present invention are readily soluble in various solvents and have molecular weight of between about 300 and about 100,000 Da and preferably between about 2,000 and about 40,000 Da. The biocompatible polymers include, but are not limited to, polyethylene glycol (PEG), polypropylene glycol (PPG), polyoxyethylene (POE), polytrimethylene glycol, polylactic acid and its derivatives, polyacrylic acid and its derivatives, polyamino acid, polyvinylalcohol, polyurethane, polyphosphazene, poly(L-lysine), polyalkylene oxide (PAO), polysaccharide, dextran, polyvinyl pyrrolidone, polyacrylamide, copolymers thereof and other nonimmunogenic polymers.

Biocompatible polymers of the present invention are intended to include not only linear polymers but also polymers as follows. Biocompatible polymers of the present invention include soluble, non-antigenic polymers linked to an activated functional group that is capable of being nucleophilically substituted through an aliphatic linker residue (U.S. Pat. Nos. 5,643,575 and 5,919,455). Also, biocompatible polymers of the present invention include multi-armed, mono-functional and hydrolytically stable polymers, having two linker fragments which have polymer arms around a central carbon atom, a residue which is capable of being activated for attachment to biologically active materials such as proteins, and side chains which can be hydrogen or methyl group, or other linker fragment (U.S. Pat. No. 5,932,462). In addition, biocompatible polymers of the present invention include polymers of branched PEG in which the functional groups of polymers are attached to biologically active materials via linker arms having reporter residues (WO 00/33881).

Among them, PEG is one of the most common biocompatible polymers of the present invention. In general, PEG is a nontoxic hydrophilic polymer having the repeating unit, HO—(CH₂CH₂O)ₙ—H. Various proteins are reported to show extended half-lives, increased solubility, increased stability, and reduced immunogenicity in plasma when being conjugated with PEG.

The range of molecular weight of PEG molecules conjugated to biologically active materials such as proteins or peptides is from about 1,000 to 100,000 Da and the toxicity of PEG over 1,000 Da is known to be very low. PEGs in the range of from 1,000 to 6,000 Da are distributed to the whole body and cleared in the kidney. Branched PEG with molecular weight of 40,000 Da are distributed in blood or organs including the liver, and metabolized in the liver.

PEG is a most preferable biocompatible polymer because PEG is commercially available in the various molecular weight ranges, each oxyethylene unit is hydrophilic to be accessible to bind 2-3 water molecules, PEG derivatives with one-terminal functional group from methoxy polyethylene glycol are easy to synthesize, PEG has very low risk of antigen-antibody reaction, and the related technology is well developed.

Biologically Active Materials

The term “biologically active molecule” or “biologically active material” means all nucleophiles conjugated with activated biocompatible polymers, and which retain at least some of their biological activity after conjugation. Preferred embodiments are directed to biologically active molecules which include hGH. The term “biological activity” used herein is not limited by physiological or pharmacological activity. In general, biologically active molecules can be isolated from nature or synthesized recombinantly or chemically, and include proteins, peptides, polypeptides, enzymes, biomedicines, genes, plasmids, or organic residues.

Human Growth Hormone

The term human Growth Hormone (hGH) as used herein encompasses human Growth Hormone and also variants of hGH such as analogs, fragments, homologs, derivatives or allelic variants of hGH which have the same function as the naturally occurring polypeptide. Growth Hormone according to the invention may be purified from human or animal sources, produced chemically or recombinantly. Preparations of hGH are commercially available. Recombinantly produced hGH may be referred to as SYNTROPIN™.

The manufacture of recombinant hGH species is well known and is taught by U.S. Pat. Nos. 6,566,328; 5,962,411 & 5,334,531 which are incorporated herein by reference. hGH to which at least one PEG has been attached may be referred to herein as “pegylated hGH”, PEG-hGH, pegylated SYNTROPIN™ or PEG-SYNTROPIN.

In preferred embodiments, a recombinant hGH is produced using a bacterial host cell. More preferably, the bacterial host cell is transfected with bacteriophage lambda which causes lysis and release of the recombinant hGH. Yet more preferably, the bacteriophage lambda is capable of delayed lysis as taught by U.S. Pat. No. 6,773,899, which is incorporated herein by reference.

The hGH protein, produced either recombinantly or by isolation from human tissue sources, may be subsequently purified by any means known in the art including, but not limited to, ammonium sulfate precipitation, column chromatography including HPLC, ion exchange chromatography and affinity chromatography, gel exclusion and the like. In preferred embodiments, a concentration step using ammonium sulfate is followed by purification using a combination of ion exchange chromatography and hydrophobic interaction chromatography. In a most preferred embodiment, a single step column chromatography with an anion exchange column, preferably, a Q sepharose FF column is used following concentration by ammonium sulfate. Preferably, the purified hGH protein has a purity of at least 80%, more preferably at least 85% and yet more preferably more than 90% purity.

Preparation of Biocompatible Polymer-biologically Active hGH Conjugates

To conjugate biocompatible polymers to a biologically active hGH, one of the end groups of polymers is converted into a reactive functional group. This process is referred to as “activation” and the product is called an “activated” polymer. For instance, to conjugate poly(alkylene oxides, PAO), one of the hydroxyl end groups of the polymer can be converted into a reactive functional group such as carbonate and activated PAO is produced, which is soluble at room temperature. This group includes mono substituted poly(alkylene oxide) derivatives such as mPEG or other suitable alkyl-substitute PAO derivatives containing C 1-4 end group.

The term “reactive functional group” used in the art and herein is the group or the residue activating biocompatible polymers to bind with biologically active hGH.

The reactive functional group of the present invention is selected from the functional groups able to react with carboxylic acid and reactive carbonyl group, for example, primary amine, or hydrazine and hydrazide functional groups (such as acyl hydrazide, carbazate, semicarbazate, thiocarbazate etc.).

The term “coupling reagent of carboxyl group” (hereinafter referred to as coupling reagent) used in the art and herein means any reagent to couple the carboxyl groups of biologically active materials such as hGH to biocompatible polymers which have been activated at the above reactive functional group.

The coupling reagents of the carboxyl group in the present invention of interest include, but are not limited to, carbodiimidyl coupling agents, for example, EDAC[N-(3-dimethyl-aminopropyl)-N′-ethylcarbodiimide hydrochloride], DIC[1,3-diisopropyl carbodiimide], DCC[dicyclohexyl carbodiimide], and EDC[1-ethyl-3-(3-dimethylamino propyl)-carbodiimide]. The preferable coupling agent for the carboxyl group is EDAC.

The method of preparing the conjugates of the present invention includes the step of reacting biologically active hGH containing nucleophiles capable of performing the substitution reaction with activated biocompatible polymers under conditions in which sufficient conjugation can be possible while retaining at least a portion of intrinsic bioactivity of biologically active molecules.

Biologically active hGH-biocompatible polymer conjugates with a ratio of 2:1 or less, preferably 1:1 are obtained by reacting the biologically active materials with a stoichiometric excess amount of polymers. For example, in the preparation of hGH-PEG, the molar ratio of biologically active hGH to PEG is in the range of from about 1:1 to 1:20, more preferably from 1:1 to 1:10. The reagents to activate carboxyl groups of biologically active materials are selected from the group as follows, but are not limited to them. For example, N-(3-dimethyl-aminopropyl)-N′-ethylcarbodiimide hydrochloride (EDAC), water soluble carbodiimide group such as 3-[2-morpholinyl-(4)-ethyl], and 5-substituted isoxazolinium salts such as p-toluene sulfonate, Woodward's Reagent K.

The molar ratio of biologically active hGH to EDAC used in the present invention is in the range of from about 1:1 to 1:50, more preferably from about 1:1 to 1:30, and most preferably from about 1:1 to 1:20. Preferably, the addition of EDAC was divided to more than 5 times, preferably 5 or 6 times rather than adding 20-fold molar excess of EDAC at once because EDAC is readily hydrolyzed in aqueous solution.

The conjugation reaction of hGH with an activated polymer is dependent on the pH of water soluble solvents functioning as a buffer. In general, the pH of reaction buffer is in the range from 2 to 5, preferably from 2.5 to 4.5. The optimum reaction condition for stabilization of these substances and reaction yield is known in the art. The suitable temperature for the conjugation reaction is in the range of 0 to 60° C. and preferably in the range of 4 to 30° C. The temperature of the solvents should not exceed the denaturation temperature of proteins or peptides. A reaction time of 10 minutes to 5 hours is preferred. The hGH conjugates prepared can be recovered and purified by ammonium sulfate precipitation, column chromatography, diafiltration or a combination of these processes.

Pharmaceutical Composition

The present invention also relates to a pharmaceutical composition comprising a therapeutically effective dose of the activated biocompatible polymer-hGH conjugate as an active ingredient.

The term “pharmaceutically acceptable” used in the art and herein means not causing allergic reaction or similar reaction when administered to humans.

The biocompatible polymer-biologically active hGH conjugate as an active ingredient of the pharmaceutical composition can be used itself or formulated in combination with pharmaceutically acceptable carriers for disease prevention and treatment.

The term “pharmaceutically acceptable carrier” used in the art and herein means pharmaceutically acceptable molecules, composition, or vehicles such as solutions, diluents, excipients, or solvents to carry the biologically active hGH from one organ or tissues to other organs or tissues. The pharmaceutical composition of the present invention can be administered by oral, local, injection or parenteral route and its formulation includes therapeutically effective doses of the biocompatible polymer-biologically active hGH conjugates as an active ingredient. The formulation for oral administration of the present invention include pills, tablets, coated tablets, granules, troches, wafers, elixirs, hard and soft gelatin capsules, solutions, syrups, emulsions, suspensions, or sprays etc. and for parenteral administration, injectable solutions, microcapsules, patches, and others are included.

The pharmaceutical formulation can be prepared according to the known method by using pharmaceutically acceptable inactive inorganic or organic additives. For example, lactose, corn starch and its derivatives, talc, or stearic acid and its salts can be used to prepare pills, tablets, and hard gelatin capsules. The additives of soft gelatin capsules and suppositories are for example, oil, wax, semi-solid or liquid polyol, and natural or solidified oil. The suitable additives for preparation of solution or syrup are for example, water, sucrose, invertase, glucose, and polyol. The suitable additives for preparation of injectable solution are water, alcohol, glycerol, polyol, plant oil etc. The injectable solution can be used as the combination of preservatives, indolent agents, solubilizers, and stabilizers. The formulation for local administration can be also used as the combination of gas, diluents, lubricants, and preservatives. The suitable additives for microcapsules or transplantation are copolymer or glycolic acid and lactic acid.

The dose of the biocompatible polymer-biologically active hGH conjugates of the present invention varies depending on the absorption rate of the hGH, solubility, patient's age, sex, condition and severity of diseases, etc. as well known in the art. In Example 4 shown below, pegylated hGH proteins (hGH) that retain up to 20% of the activity of native hGH and have significantly larger (10-fold higher) half-lives in the circulation of animals are shown. In preferred embodiments, PEG-hGH retains at least 1%, more preferably 5%, yet more preferably 10% and yet more preferably 15% of the native activity. Preferred embodiments retain 10-20% of the activity of the native hGH.

In preferred embodiments, pegylated hGH according to the invention has at least 3 fold, preferably at least 5 fold, yet more preferably at least 7 fold and yet more preferably at least 10 fold greater half lives in circulation in vivo compared to native hGH.

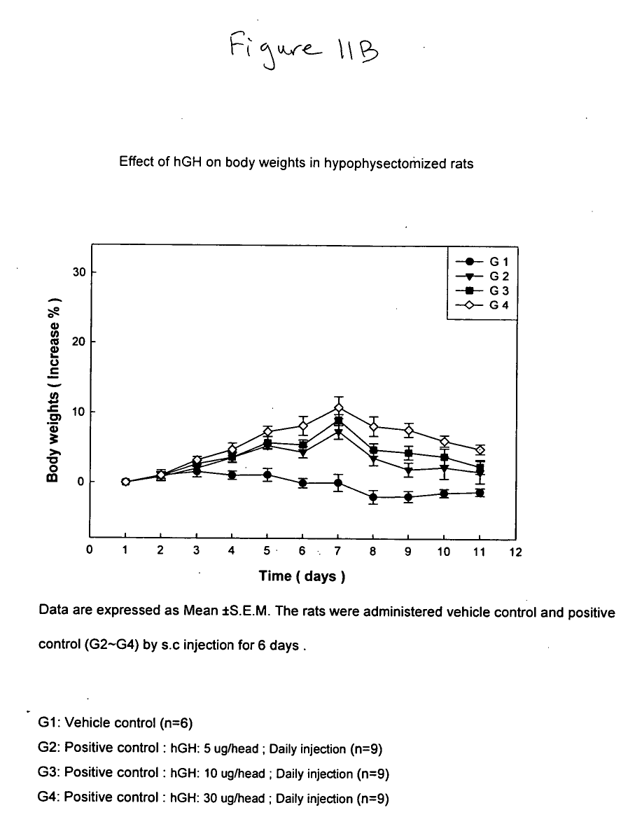

PEG-hGH clearly has the potential to show clinical utility combined with a much easier form of administration. In preferred embodiments, administration of pegylated hGH is less than daily, preferably no more than 5 times per week, more preferably no more than 4 times per week, yet more preferably no more than 3 times per week, yet more preferably no more than 2 times per week, and yet more preferable no more than one time per week. As shown in an animal weight gain assay in FIG. 10, a weekly injection of pegylated hGH or SYNTROPIN™ (recombinant hGH) is equivalent in potency to daily injections of native hGH, which is the component of all brands currently in the marketplace. According to the Human Growth Foundation, it is estimated that 10,000-15,000 children in the United States have growth failure due to growth hormone deficiency. Clearly this demonstrates a need for a slow release form of hGH.

Particularly, the administration of biocompatible polymer-biologically active hGH conjugates of the present invention reduces the injection intervals from daily or once per two days to weekly or biweekly injection. Therefore, the toxicity and site effects of drugs by frequent administration are reduced substantially.

Therapeutic Uses

Any condition amenable to treatment by unmodified growth hormone (GH) may be treated with PEG-hGH according to embodiments of the invention. In particular, PEG-hGH according to embodiments of the invention may be used to treat children with growth hormone (GH) deficiency, generally defined as a growth in height of less than 2 inches per year, although more extensive testing confirms a growth hormone deficiency. This GH deficiency may be due to a congenital problem, a tumor, infection or radiation treatment such as for tumors to the head and neck.

PEG-hGH according to embodiments of the invention may also be used for treatment of the results of interuterine growth restriction. In some cases, an infant may be small for its gestation time due to maternal nutrition, infectious disease, environment, excess maternal alcohol consumption or other factors. Administration of PEG-hGH allows children suffering from this disorder to catch up to their peers in growth.

PEG-hGH according to embodiments of the invention may be used in treatment of chronic renal insufficiency in children as hGH is effective in stimulating growth.

PEG-hGH according to embodiments of the invention may be used to treat Turner syndrome. Although Turner syndrome is not caused by GH deficiency, administration of GH may allow girls afflicted with Turner syndrome to reach a normal height. PEG-hGH according to embodiments of the invention may be used in treatment of symptoms of Prader-Willi syndrome to increase growth and lean body mass and decrease body fat.

PEG-hGH according to embodiments of the invention may be used to treat idiopathic short stature, that is, height that is well below average for a child's age and sex. For children who do not present with a growth hormone deficiency and are normal physically, but more than two standard deviations below normal height, PEG-hGH according to embodiments of the invention may be used to increase height in these children.

Children who have growth hormone therapy as children often benefit from this therapy as adults as well. As adults they may not need to grow taller, but may still be deficient in growth hormone which leads to excess fat, decreased muscle mass and low vitality. In addition, some adults who did not have growth hormone therapy as children may produce insufficient amounts of growth hormone as adults. Symptoms of a GH deficiency include increased fat around face and abdomen, low level of lean body mass, bone loss, thinning skin with fine wrinkles, poor sweating or body temperature regulation, low interest in sex, sleep problems, poor muscle strength, poor exercise performance, high cholesterol levels, production of too much insulin and depression. PEG-hGH according to embodiments of the invention may be used in treatment programs for these patients.

GH has been FDA approved for use in adults for treatment of wasting syndrome due to AIDS, burns or traumatic injuries. PEG-hGH according to embodiments of the invention may be used to treat these conditions.

GH has also been shown to be useful to combat effects of aging. As part of the aging process, the production of GH diminishes. GH depletion is marked by the usual signs of aging, which includes increased body fat (especially around the waist), reduced vitality, decreased muscle mass, increased blood pressure and cholesterol and poor general health. PEG-hGH according to embodiments of the invention may be used to treat these symptoms.

Conjugated hGH as described above may be conveniently provided to the patient or health care practitioner as a kit. The kit includes the hGH conjugate, preferably in a pre-measured dose form. The kit preferably includes one or more containers of hGH conjugate as a pre-measured dose.

The hGH conjugate would be provided in lyophilized form or in a pharmaceutically acceptable carrier. If the hGH conjugate is provided in lyophilized form, the kit would preferably also include a pharmaceutically acceptable carrier for reconstitution of the conjugate.

In preferred embodiments, the kit would include a delivery device for delivering the hGH to the individual being treated. The delivery device is preferably a syringe. In some embodiments, the kit may include a syringe preloaded with the pre-measured dose of hGH conjugate.

In some preferred embodiments, the kit may also include any of a skin antiseptic for treatment of skin before delivery using the delivery device or syringe, bandaging material and instruction sheet.

The following examples further describe and demonstrate embodiments within the scope of the present invention. The examples are given solely for the purpose of illustration and are not to intended limit the present invention, as many variations thereof are possible without departing from the spirit and scope of the invention.

### EXAMPLES

### Example 1

### Production of Human Growth Hormone by Phage-dependent Method

Recombinant hGH was prepared essentially as taught in U.S. Pat. No. 6,773,899 which is incorporated herein by reference. Cultures of Escherichia coli BL21(DE3) (NOVAGEN) were transformed by a plasmid which contains one copy of a chemically synthesized gene encoding human growth hormone (SEQ ID NO: 4). The translated amino acid sequence is shown as SEQ ID NO: 5. Cultures of BL2 1 (DE3) contain a single copy of the gene for T7 RNA polymerase under the control of the inducible lac UV5 promoter in the bacterial genome (Studier et al. (1986) J. Mol. Biol. 189: 113-130). Into the plasmid pET-24a(+) (NOVAGEN) was inserted the human growth hormone gene under the control of the T7 promoter. Expression of the human growth hormone gene begins only after the appearance of T7 RNA polymerase in the cells which is mediated through the induction of the lac UV5 promoter by IPTG.

The transformed cultures of E. coli BL21(DE3) were grown with shaking at 37° C. in LB medium, containing 50 μg/ml kanamycin, to a density of 2×10⁸ cells/ml. Then the cells were infected with phage λ cI₈₅₇ Qam₁₁₇ Ram₅₄ at a multiplicity of about 10 phage bodies per 1 bacterial cell and cultivated with shaking at 23° C. for about 14 hour. Simultaneously with phage, 1 mM IPTG was introduced into the medium.

Phage λ cI₈₅₇ Qam₁₁₇ Ram₅₄ was prepared from lysogenic cultures of E. coli RLMI, which were grown in LB medium at 28° C. with intensive aeration to a density of approximately 1×10⁸ cells/ml. The lysogenic culture was warmed to 43° C. and incubated for 20 minutes to inactivate cI repressor. The temperature was then decreased to 37° C. and after 60-70 minutes the bacterial cells underwent lysis, with phages being formed at 1-2×10¹⁰ PFU/ml.

After incubation with the phage-infected cells for 14 hours, debris was removed from the culture medium by centrifugation to produce conditioned media.

### Example 2

### Purification of Native hGH-3 Step Method

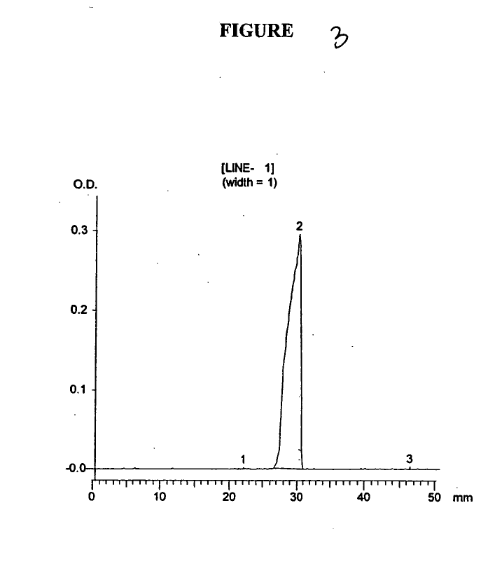

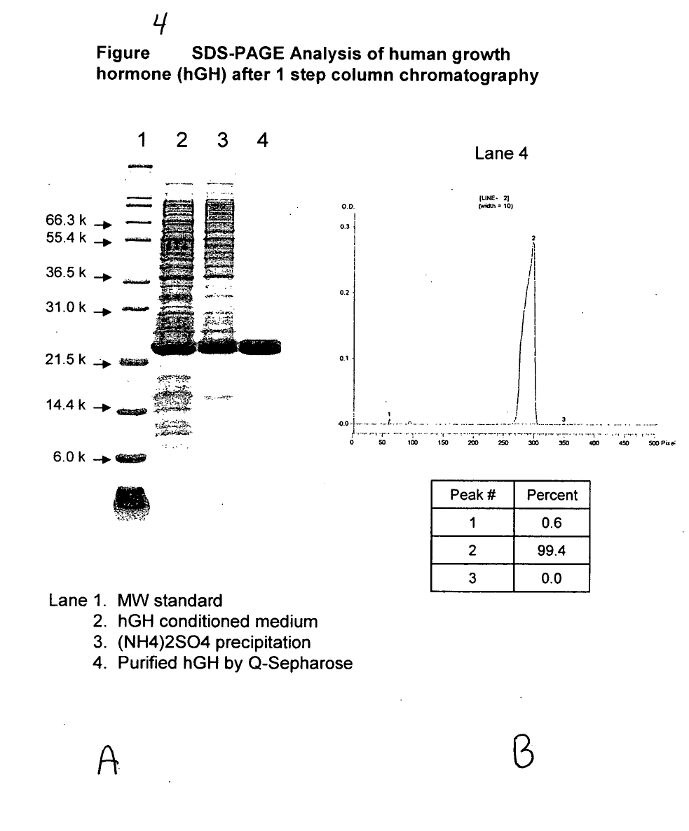

The conditioned media (bacterial growth media after bacterial lysis containing the released protein) from Example 1 was purified with ammonium sulfate precipitation, followed by purification on Q-Sepharose and Phenyl Sepharose columns. FIG. 2, displays an SDS-PAGE gel of the drug substance obtained after each successive purification step. The soluble, biologically-active growth hormone product in conditioned medium is shown in lane 2 (FIG. 2). The drug substance is then subjected to the 3-step purification procedure of ammonium sulfate precipitation, followed by successive chromatography steps on Q-Sepharose and Phenyl-Sepharose. The purified, final drug substance depicted in Lane 5 of FIG. 2, was judged to be greater than 99% pure by a densitometric scan of this lane as shown in FIG. 3. This process produces a human growth hormone product of high purity with biological activity equivalent to an international growth hormone standard. It was used in subsequent pegylation studies.

### Example 3

### Preparation of Native hGH—One Column Method

In some studies, a one column purification method was employed. This protein purification procedure for human growth (hGH) requires a single column chromatography step. The first ammonium sulfate precipitation step is a concentrating step prior to running the column chromatography.

(NH₄)₂SO₄ Precipitation Step

A 3 liter fermentation run of hGH produced by the method described in Example 1 was frozen and the lysed bacterial culture was thawed at 4° C. and clarified by centrifugation at 16000 g to obtain conditioned medium. An equal volume of saturated (NH₄)₂SO₄ solution was pumped into the conditioned medium with stirring to a final concentration of 50% saturation and the mixture was stirred for an additional 1 hour. The precipitate was collected by centrifugation at 16000 g for 1 hour. The pellets were dissolved in 20 mM Tris.Cl pH 8.0 the volume of which is 1/10 of the conditioned medium. The solution was dialyzed against 20 mM Tris.Cl pH 8.0. The buffer exchanged solution was clarified by centrifugation or filtered to remove any precipitate.

Q-Sepharose FF Column Chromatography Step

The buffer exchanged sample was applied to a Q-Sepharose FF column equilibrated with 20 mM Tris.Cl, pH 8. The column was washed with 20 mM Tris.Cl pH8.0 until the absorption at A280 nm reached the baseline. The column was then washed with 70 mM NaCl-20 mM Tris.Cl pH8.0 until the absorption at A280 nm reaches baseline. The hGH was eluted with 120 mM NaCl-20 mM Tris.Cl pH8.0 and the elution peak was collected. The purity of the eluate was determined by SDS-PAGE and HPLC. Typically, over 95 % pure hGH was obtained.

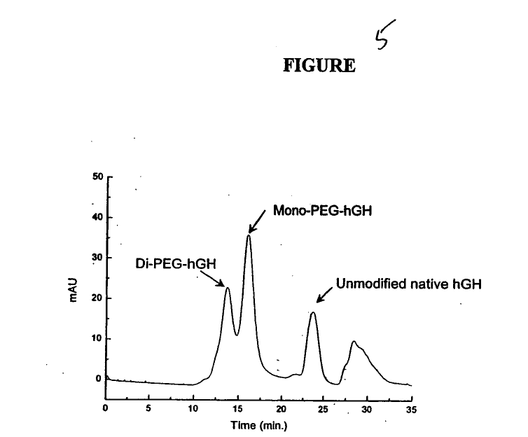

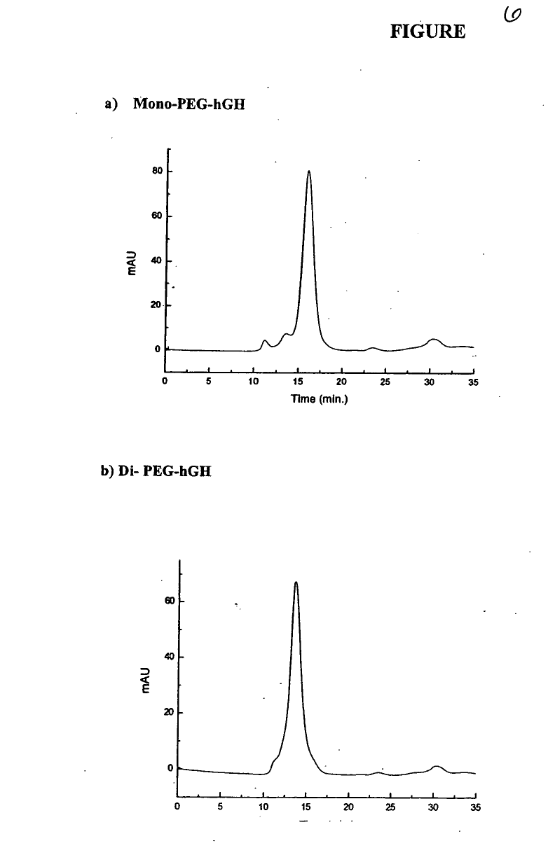

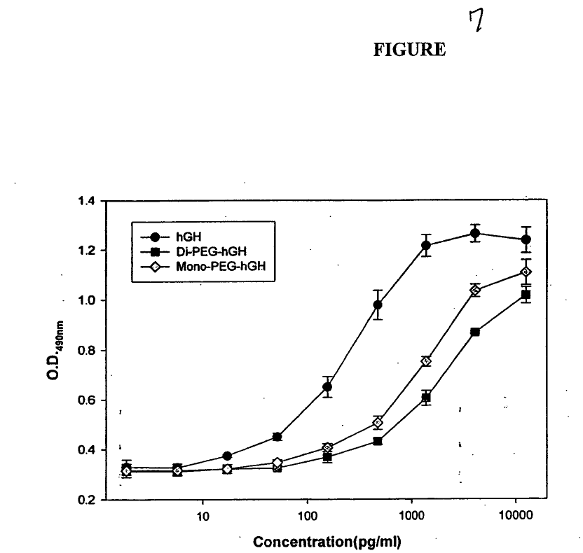

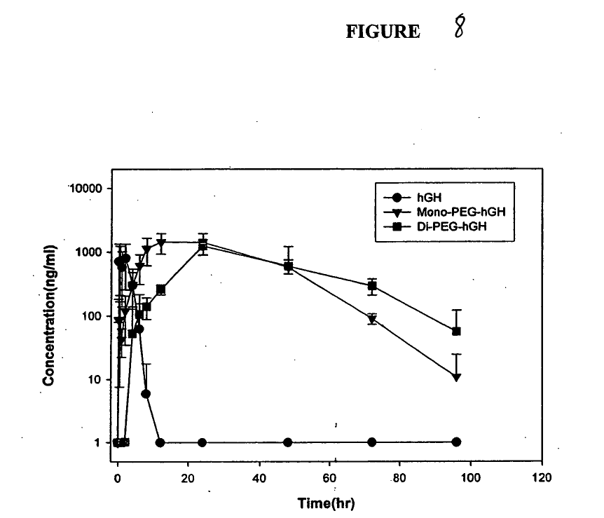

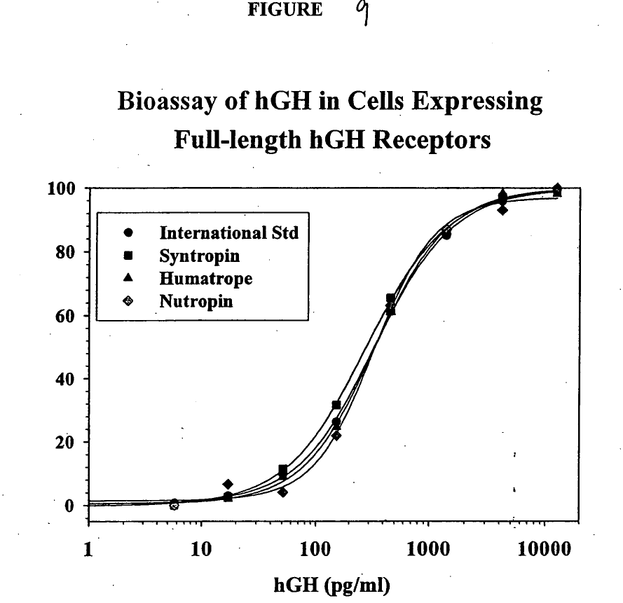

FIG. 4A shows the purity of the hGH preparation at the various steps of purification by an SDS-PAGE gel. FIG. 4A shows the actual gel image with Lane 1 representing molecular weight standards, and Lanes 2-4 showing the relative purity of the hGH preparation through the purification steps. Lane 2 is an aliquot of the conditioned media or phage lysate obtained after the fermentation run; Lane 3 is after precipitation of the conditioned media by ammonium sulfate; and Lane 4 is an aliquot of the hGH after the single Q-Sepharose chromatography step.

FIG. 4B shows a densitometric analysis of Lane 4 of the SDS-PAGE gel which calculates the relative purity of the hGH. As can be seen the hGH was judged to be 99.4 per cent pure after the single column chromatography step.

### Example 4

### Preparation of Pegylated hGH

Human Growth Hormone, purified by one of the methods described above was pegylated using the methods as described in U.S. application Ser. No. 10/947,513, incorporated herein by reference. Alternatively, hGH may be obtained from commercial sources. Briefly, 1 mg of hGH was dialyzed (Centricon-10, Amicon, USA) against 50 mM MES buffer solution (pH 3.0) to a final concentration of 2 mg/ml. To this protein solution, mPEG-hydrazide (Hz) (ISU Chemical, Korea, 0.0005 mmol) was added and followed 20-fold molar excess of EDAC in solution prepared by dissolving 2 mg of EDAC in 20 ul of d-H2O. EDAC was used at a 15-fold molar excess to activate PEG. The reaction was carried out using either PEG (20000) or PEG (30000). The reaction was carried out for 1 hour at room temperature (20-25° C.) with stirring. After 1 hour, unreacted hGH and excess reagent were removed by size exclusion column or ion-exchange column. In preferred embodiments, the amount of EDAC ranges from 20 to 50-fold molar excess and mPEG-Hz from 10 to 20-fold molar excess. Mono-PEG-hGH (one PEG attached to one hGH molecule, Lot No BPM#04-003) and di-PEG-hGH (two PEGs attached to one hGH molecule, Lot No BPM#04-004) were separated by HPLC using a size-exclusion column. The purified PEG-hGH fractions were stored in PBS solution at 4-8 C. until further analysis and used to evaluate PEG-hGH in vitro biological activity and half-life in rats.

The reaction of hGH with activated PEG derivatives was verified by HPLC using a size-exclusion column monitored at 220 nm as shown in FIG. 5. The concentration of PEG-hGH was determined by O.D. at 280 nm using UV-VIS spectrophotometer. Approximately 48 %, 31 %, and 21 % of mono-PEG-hGH, di-PEG-hGH, and unreacted hGH were produced, respectively. Each fraction was then purified by size-exclusion column and verified on HPLC as shown in FIG. 6A (mono-PEG-hGH) and 6B (di-PEG-hGH). The purity of each PEG-hGH samples was determined to be >95 %.

Biological Activity of PEG-hGH: PK Study of PEG-hGH in Rats

The biological activity of mono- and di-PEG-hGH was determined by cell proliferation assay and compared with native hGH (Product of Phage Biotech). Mono- and di-PEG-hGH were administered to 7-week old Sprague-Dawley rats (at least 5 rats per each group) weighing 220-240 g with a dose of 200 ug/kg by s.c. injection, respectively. Native hGH was used as a control. The blood was withdrawn at a time interval of 0, 10 min, 30 min, 1 hr, 2 hr, 4 hr, 6 hr, 8 hr, 12 hr, 24 hr, 48 hr, 72 and 96 hr. post injection. The serum samples were obtained by centrifugation at 12000 rpm and stored at −20 C. for further analysis.

FIG. 7 shows the biological activity of the mono-and di-PEG-hGH as well as native hGH measured by cell proliferation assay. The bioassay is a cell proliferation assay, where hGH stimulates the proliferation of BaF3 cells, which have been stably transfected with the full-length human growth hormone receptor. This cell line termed Baf-B03 B2B2 has been extensively characterized (Behncken S N, et al. 1997. J Biol Chem 272:27077-27083) in terms of its response to hGH. The activity of mono- and di-PEG-hGH was determined to be 15±5 % and 8±2 %, respectively.

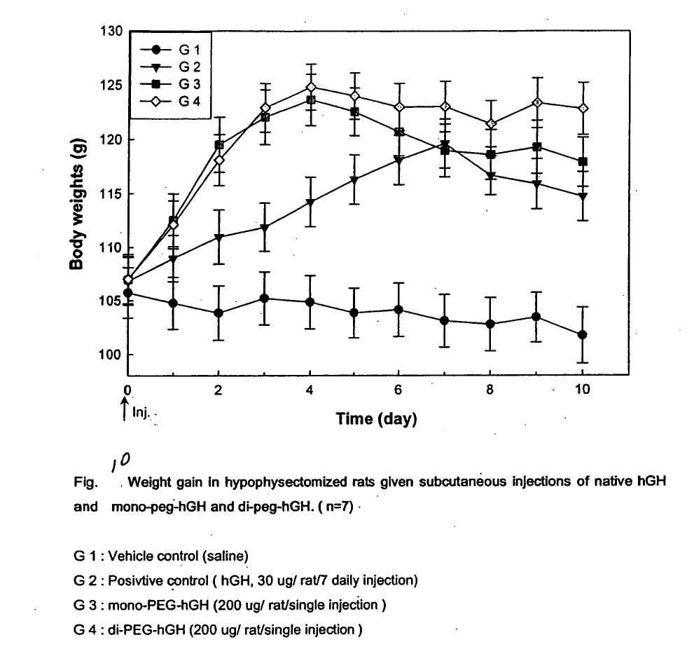

The pharmokinetic (PK) study of PEG-hGH was performed by measuring the amount of hGH in serum samples using ELISA assay compared with native hGH (Phage Biotech). FIG. 8 shows the PK study of PEG-hGH and compared to native hGH. It was shown that native hGH was cleared from the blood within 5 hours post injection whereas both mono- and di-PEG-hGH were detected after 72 hours post injection.

We observed that mono- and di-PEG-hGH retained 15±5 % and 8±2 % of biological activity, respectively as compared to native hGH. The PK study, however, shows that PEG-hGH was cleared much slower than native hGH in rats. Therefore, the PEG-hGH samples of this study can provide a new sustained released drug of hGH.

### Example 5

### Bioassay

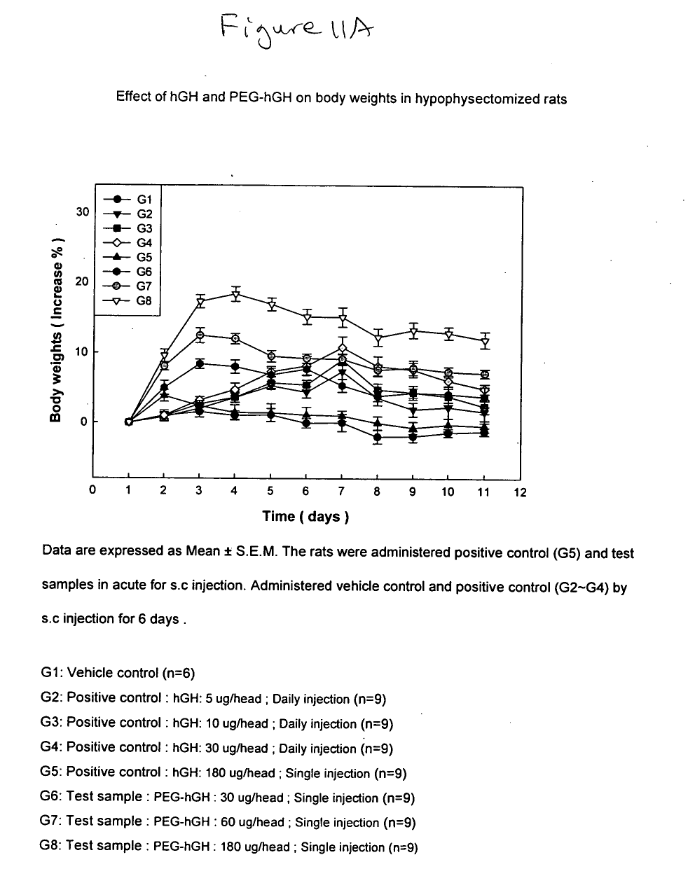

The activity of the preparation described above was tested by the cell proliferation assay with BaF3 cells and compared to commercially available hGHs. SYNTROPIN™, NUTROPIN®, and HUMATROPE® are commercially available hGH forms. A representative standard curve is shown in FIG. 9 where similar dose response curves are seen with four commercially available hGHs. The specific activity of hGH prepared as described above compares favorably with commercially available hGH. As expected, pegylation of the native SYNTROPIN™ resulted in a loss of biological activity, with a greater activity loss occurring as one attaches more PEG groups to the native hGH.

TABLE 1
Potencies (IU/mg) of hGH Preparations Tested
in the Cell Proliferation Assay

| Samples | Specific Activity (IU/mg) |
|---|---|
| International Std | 3.00 |
| SYNTROPIN ™ | 3.11 |
| NUTROPIN ® | 3.03 |
| HUMATROPE ® | 3.03 |
| Mono-PEG | 0.46 |
| SYNTROPIN | |
| Di-PEG | 0.25 |
| SYNTROPIN | |

### Example 6

### Effect of PEG-hGH on Body Growth in Male Hypophysectomized Rats

A second bioassay utilized was the classical rat weight gain assay (Roswall E C, et al. 1996. Biologicals 24: 25-39) where the weight of 4-5 week old hypophysectomized rats (Orient, Inc. 143-1 Sangdaewon-dong, Sung-Nam, Kyunggi-do) were monitored over a 28 day period, following subcutaneous hGH injections, once daily, for the first 7 days, or one injection at day 1 of the mono- or di-PEG-SYNTROPIN. Remarkably, and unexpectedly given the rather low potency of the PEG-SYNTROPINS in the cell-based bioassay (Table 1) , the pegylated SYNTROPINS showed equivalent activity with native hGH when injected only once a week versus daily injections for native hGH (see FIG. 10). It can be seen in FIG. 10 that at 7 days, rats given daily injections of native hGH weighed approximately the same as rats given a single injection of either the mono-PEG-SYNTROPIN or the di-PEG-SYNTROPIN. Also unexpected was the rate of weight gain in the animals administered PEG-SYNTROPINS versus those animals receiving native SYNTROPIN. In FIG. 10 it can be seen that the rats given a single dose of either mono-PEG-hGH or di-PEG-hGH gained weight at a significantly faster rate over the first 4 days than those animals receiving a daily injection of native hGH. It was not until day 7 that the animals receiving native hGH were able to “catch up” in body weight with the animals receiving the pegylated growth hormones.

The effect of PEG-hGH on body growth in male hypophysectomized rats was further investigated with different doses. The 4-5 week-old hypophysectomized rats were purchased, stored for 5 days, administered PEG-hGH(G6˜G8) by s.c. once, and determined the body weights daily for 11 days. A saline solution as a negative control (Vehicle control, GI) and native hGH (Positive control, G2˜G4) were administered by s.c. every day for 6 days (Day 1-Day 6) and the body weight of each rat was measured for 11 days. Also, a high dose (180 μg) hGH(G5) was administered by s.c. once and the body weight was measured to compare with other groups.

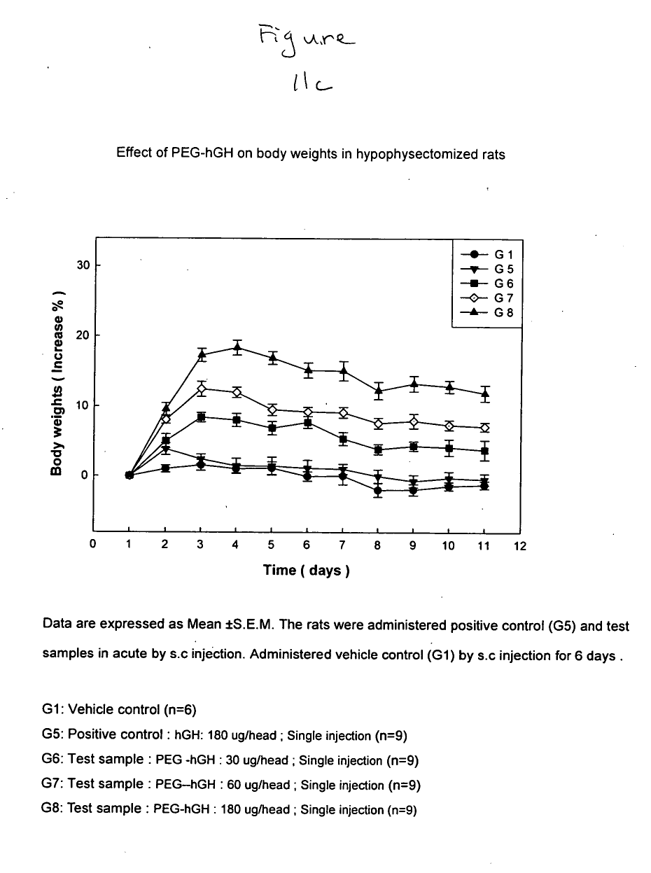

No unusual symptoms were observed as a result of any of the treatments and no significant change in body weight was observed in the saline control (G1, FIGS. 11 A-C) or the hGH administered group (G5, FIGS. 11A, 11C).

From this study, it was found that the body weights of rats for the positive control, G2, G3 and G4, were increased by 7.37 %, 9.03 %, and 10.79 %, respectively, during the daily administration of hGH (FIGS. 11A, 11B), while the body weights of rats for the PEG-hGH rats, G6, G7 and G8, were increased by 8.42 %, 12.48 %, and 18.37 %, respectively, after single administration of PEG-hGH (FIGS. 11A, 11C). This result shows that the increase of body weight is proportional to the amount of hGH or PEG-hGH administered. However, one bolus administration of high dose of hGH (180 ug/rat) produced only a small and transitory response (G5, FIGS. 11A, 11C), whereas a single administration of PEG-hGH gave a much larger and more sustained response.

It was observed that the single injection of PEG-hGH to hypophysectomized rats enhanced the body weight notably and fairly maintained the increased body weight at least for 6 days. The daily injection of hGH enhanced the body weight but the body weight began to decrease as soon as the administration of hGH was stopped. It was also observed that the single bolus high dose injection of hGH (G5) enhanced body weight by <4 % the day after administration followed by decreased body weight to as low as negative control indicating that the continuous injection of native hGH was necessary to enhance body weight continuously for the non-pegylated hGH samples.

Dose dependent body weight gain was observed for hGH as well as PEG-hGH. In other words, the body weight gain was proportional to the amount of hGH or PEG-hGH administered in hypophysectomized rats.

This study shows that single injection of PEG-hGH enhanced body growth and fairly maintained body weight at least for 6 days in hypophysectomized rats whereas daily injection of non-pegylated hGH for 6 days was necessary to maintain the enhanced body weight of rats continuously. PEG-hGH as a weekly injectable drug is a promising alternative to daily injections of hGH.

Thus, while there have been described the preferred embodiments of the present invention, those skilled in the art will realize that other embodiments can be made without departing from the spirit of the invention, which includes all such further modifications and changes as come within the meaning, true scope of the claims set forth herein and equivalents thereof. The above examples further describe and demonstrate embodiments within the scope of the present invention. The examples are given solely for the purpose of illustration and are not to be construed as limitations of the present invention, as many variations thereof are possible without departing from the spirit and scope of the invention.

What is claimed is:

1. A conjugate of biocompatible polymer-human Growth Hormone (hGH), wherein the biocompatible polymer is conjugated to a carboxyl group of hGH at a molar ratio of 2:1 or less.

2. The conjugate according to claim 1, wherein the biocompatible polymer is selected from the group consisting of PEG-20000 and PEG-30000.

3. A pharmaceutical composition comprising a pharmaceutically acceptable amount of the conjugate according to claim 1 and a pharmaceutically acceptable carrier.

4. The conjugate of claim 1, wherein the carboxyl group is the C-terminus of hGH.

5. The conjugate of claim 1, wherein the activity of the conjugate is 10-20% of the activity of an unconjugated hGH protein.

6. The conjugate of biocompatible polymer-hGH according to claim 1, wherein the biocompatible polymer is conjugated to the carboxyl group of the hGH at a molar ratio of 1:1.

7. The conjugate of claim 6, wherein the biocompatible polymer is PEG.

8. A method of preparing a conjugate of biocompatible polymer-hGH comprising:
(a) providing a purified hGH protein;
(b) activating a biocompatible polymer with the stepwise addition of a coupling reagent; and
(c) conjugating the activated biocompatible polymer to a carboxyl group of the hGH at a molar ratio of 2:1 or less,
wherein the molar ratio of the hGH to the activated biocompatible polymer is 1:1 to 1:20, the ratio of the hGH to the coupling reagent is 1:1 to 1:50, and pH is in the range of 2 to 5.

9. The method according to claim 8, wherein the biocompatible polymer is activated with a reactive functional group which is able to react with a carboxylic acid and/or a reactive carbonyl group.

10. The method according to claim 8, wherein the biocompatible polymer is selected from the group consisting of PEG-20000 and PEG-30000.

11. The method of claim 8, wherein the carboxyl group is the C-terminus of hGH.

12. The method of claim 8, further comprising providing the purified hGH by the steps of:
(i) producing hGH in a recombinant host;
(ii) concentrating hGH using ammonium sulfate; and
(iii) purifying the concentrated hGH by anionic ion exchange chromatography.

13. The method of claim 12, wherein the chromatography is performed as a single step.

14. A method of treating growth failure or growth retardation by administering an effective amount of the conjugate of claim 1 to a patient in need thereof.

15. The method of claim 14, wherein the biocompatible polymer is PEG.

16. The method of claim 15, wherein the PEG-hGH is conjugated at a molar ratio of 1:1.

17. The method of claim 14, wherein the conjugate has 10-20% of the activity of unconjugated hGH protein.

18. The method according to claim 14, wherein the growth failure or growth retardation is due to hormone deficiency, chronic renal disease, Turner's syndrome, cachexia or AIDS wasting.

19. The method according to claim 14, wherein the conjugate is administered in combination with a pharmaceutically acceptable carrier.

20. The method according to claim 14, wherein the administration is done by injection.

21. The method according to claim 14, wherein the administration is oral.

22. The method according to claim 14, wherein the composition is administered no more than twice per week to the patient in need thereof.

23. A method of treating short stature in children by administering an effective amount of a composition comprising PEG-hGH to a patient in need thereof at a frequency of no more than twice/week, wherein the PEG is conjugated to a C-terminus carboxyl group of hGH at a molar ratio of 1:1 and the PEG-hGH has 10-20% of the activity of unconjugated hGH.

24. A method of treating adverse effects associated with aging selected from the group consisting of decrease in lean muscle, increase in blood pressure, increase in cholesterol, increase in body fat, loss of skin tone, and decrease in bone density by administering an effective amount of a composition comprising PEG-hGH to a patient in need thereof, wherein the PEG is conjugated to a carboxyl group of hGH at a molar ratio of 1:1.

25. A kit comprising:
the conjugate of claim 1 in lyophilized form;
a pharmaceutically acceptable carrier for reconstitution of the conjugate; and
a delivery device for delivery of the reconstituted conjugate to a patient in need thereof.

26. The kit of claim 25, further comprising:
a skin antiseptic; and
an instruction sheet.

27. The kit of claim 26, wherein the instruction sheet directs administration of the biocompatible polymer-hGH conjugate composition no more than twice per week to the patient in need thereof.

28. A kit comprising the conjugate of claim 1 preloaded in a syringe.


SEQUENCE LISTING (5 sequences)

SEQ ID NO: 1 - protein, 192 aa, Homo sapiens
PFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF

SEQ ID NO: 2 - DNA, 1822 bp, Artificial Sequence
  Chemically synthesized sequence for Human Growth Hormone using codons preferred for expression in E. coli
ggagcttctaaattatccattagcacaagcccgtcagtggccccatgcataaatgtacacagaaacaggtgggggcaacagtgggagagaaggggccagggtataaaaagggcccacaagagaccggctcaaggatcccaaggcccaactccccgaaccactcagggtcctgtggacgctcacctagctgcaatggctacaggtaagcgcccctaaaatccctttgggcacaatgtgtcctgaggggagaggcagcgacctgtagatgggacgggggcactaaccctcaggtttggggcttctgaatgagtatcgccatgtaagcccagtatggccaatctcagaaagctcctggtccctggagggatggagagagaaaaacaaacagctcctggagcagggagagtgctggcctcttgctctccggctccctctgttgccctctggtttctccccaggctcccggacgtccctgctcctggcttttggcctgctctgcctgccctggcttcaagagggcagtgccttcccaaccattcccttatccaggctttttgacaacgctatgctccgcgcccatcgtctgcaccagctggcctttgacacctaccaggagtttgtaagctcttggggaatgggtgcgcatcaggggtggcaggaaggggtgactttcccccgctgggaaataagaggaggagactaaggagctcagggtttttcccgaagcgaaaatgcaggcagatgagcacacgctgagtgaggttcccagaaaagtaacaatgggagctggtctccagcgtagaccttggtgggcggtccttctcctaggaagaagcctatatcccaaaggaacagaagtattcattcctgcagaacccccagacctccctctgtttctcagagtctattccgacaccctccaacagggaggaaacacaacagaaatccgtgagtggatgccttgaccccaggcggggatgggggagacctgtagtcagagcccccgggcagcacaggccaatgcccgtccttcccctgcagaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatgggggtgggggtggcgctaggggtccccaatcttggagccccactgactttgagagctgtgttagagaaacactgctgccctctttttagcagtccaggccctgacccaagagaactcaccttattcttcatttcccctcgtgaatcctctagcctttctctacaccctgaaggggagggaggaaaatgaatgaatgagaaagggagggagcagtacccaagcgcttggcctctccttctcttccttcactttgcagaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttctagctgcccgggtggcatccctgtgacccctccccagtgcctctcctggccttggaagttgccactccagtgcccaccagccttgtcctaataaaattaagttgcatcattttgtctgactaggtgtcctctataatattatggggtggaggggggtggtttggagca

SEQ ID NO: 3 - protein, 217 aa, Homo sapiens
MATGSRTSLLLAFGLLCLPWLQEGSAFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF

SEQ ID NO: 4 - DNA, 579 bp, Artificial Sequence
  Chemically synthesized sequence for Human Growth Hormone using codons preferred for expression in E. coli
atgtttccgacgatcccgctgtcccgcctttttgataacgcgatgctgcgtgcacatcgtctgcaccagctggcgtttgacacctaccaggaattcgaagaggcttacattccgaaagagcagaaatactctttcttgcagaacccacaaaccagcctgtgtttcagcgaatccatcccgactccttccaatcgtgaagagacccaacagaagagcaacctggagttgttgcgtatcagcctgctgctgattcagtcatggctggaaccggtgcagtttttacgcagcgtcttcgccaatagcctggtatacggcgccagcgattcaaacgtctatgatctgctgaaggacctggaagaaggcattcagacgctgatgggtcgcctcgaagacggttctccgcgcacgggtcaaatctttaaacaaacctactctaaattcgacactaactcgcataatgatgatgcgttgctgaagaactatggcctgctgtactgttttcgtaaagatatggataaagttgaaacctttttacgtattgtgcagtgccgttctgtggaaggcagttgcggcttctaa

SEQ ID NO: 5 - protein, 192 aa, Homo sapiens
MFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF